(19) United States
(12) Patent Application Publication
(10) Pub. No.: US 2020/0305793 A1
(43) Pub. Date: Oct. 1, 2020

(54) SMART MONOTORING AND SELF-HEATING MUTLI-LAYER BELLY BAND FOR SOOTHING INFANTS

(71) Applicants: Souheila Al-Jadda, Irvine, CA (US); Aadel Al-Jadda, San Carlos, CA (US)

(72) Inventors: Souheila Al-Jadda, Irvine, CA (US); Aadel Al-Jadda, San Carlos, CA (US)

(73) Assignee: Memeeno Inc, Irvine, CA (US)

(21) Appl. No.: 16/829,105

(22) Filed: Mar. 25, 2020

Related U.S. Application Data
(63) Continuation of application No. 62/825,270, filed on Mar. 28, 2019.

(51) Int. Cl. A61B 5/00; A61F 7/02; A61B 5/0205

(52) U.S. Cl. CPC A61B 5/6823; A61F 7/02; A61B 5/02055; A61B 5/742; A61F 2007/0022; A61B 5/7405; A61B 5/6831; A61B 5/0022; A61B 5/746

(57) ABSTRACT

A self-heating belly band for wrapping around an infant is provided. The belly band includes a multi-layer fabric construction that captures the infant's body heat and then reuses it to warm the infant's stomach and internal organs thereby providing the infant relief. The belly band also provides pressure on the infant's waist to provide the infant a cuddling, soothing, and comfort feel. Hook and loop fastener strips on top and bottom layers of the belly band allow wrapping and tightness adjustment around the infant's stomach.

The belly band also includes a plurality of monitoring system that allows medical monitoring of the baby's vital signs. The monitoring system uses ultrasonic sound or infrared light to alert a user when the baby's vital sign increases or decrease from a preselected threshold. The monitor, along with sensors is used to measure blood pressure, heartbeat, temperature, oxygen level, as well as movement. Connected to a mobile phone through an API and a hub, the caregiver is alerted as soon as an emergency occurs or when any of the preselected parameters are exceeded. The mobile API also allows the use to configure settings and receive baby's vitals on demand.

### CONTINUATION INFORMATION

This application takes priority from an earlier filed U.S. Provisional Patent Application No. 62/825,270, titled “A SMART MONITORING AND SELF-HEATING MULTI-LAYER BELLY BAND FOR SOOTHING INFANTS,” filed on Mar. 28, 2019, with Souheila Al-Jadda as the inventors. Application No. 62/825,270 is incorporated herein by reference.

### COPYRIGHT NOTICE

A portion of the disclosure of this patent document contains material that may be subject to copyright protection. The copyright owner has no objection to the facsimile reproduction by anyone of the patent document or the patent disclosure as it appears in the Patent and Trademark Office patent file or records, but otherwise reserves all copyright rights.

### TECHNICAL FIELD

The present application relates to a smart multi-layered self-heating belly band for soothing infants. More particularly, the present application is directed to soothing colicky, premature, and gassy babies through application of heat and pressure on their stomachs by applying wrapping the multi-layered self-heating belly band around their waist. The present invention also is directed to remote monitoring of an infant through a mobile application. The remote monitoring includes communications and data exchange between a monitor connected to the belly band, a hub, and an electronic device capable of downloading a mobile application.

### BACKGROUND

Adjusting to the outside world after having been in their mother's tummy can be tough for a new baby. Adding to the challenge is feeding from mother's milk, manufactured or formula milk with various ingredients, or cow's milk, and having to digest the milk while their digestive systems are still developing. If an infant is born premature or is colicky, that adds an additional layer of discomfort and leads to a crying or unhappy and uncomfortable baby.

The National Center for Health Statistics states that roughly 12 percent of all babies are born premature. Digestive problems, gassiness, constipation, Gastro-esophageal reflux, and stomach soreness are common for these early term babies. Unable to deal with the discomfort, the babies end up being restless, not being able to sleep, or constantly crying. Solutions prescribed by physicians include tummy exercise, changing feeding products, or adding thickeners to milk—which may or may not work.

Colic is common among babies. Some physicians diagnose an infant as colicky if there is persistently crying for more than 3 hours in a day and others use a different measuring scale. In brief, infantile colic refer to functional gastro-intestinal disorder, also commonly referred to as FDIC. Colic can affect up to 40% of children and babies with colic take longer and are much harder to soothe. Many times, the babies cry themselves to exhaustion and then fall asleep, only to be woken up again mid-sleep due to stomach cramping or indigestion issues.

Exercising, taking medications, rubbing the baby's back, placing a warm heating bottle or hot water pad on the baby's tummy, or giving the baby a pacifier to stop crying are the typical solutions to addressing the above-mentioned problems. Other solutions include gas relief drops, gripe water, disposable catheters for rectal insertion to relive gas, or elevated sleep pillows. The present solutions have several drawbacks and do not soothe the baby or address the root problem. For example, solutions such as hot water bottle, heat pad, or exercise needs constant monitoring and attention and is laborious. Pacifiers can be ineffective as they simply quiet the baby but the underlying stomach or indigestion problems still persists. Further, many babies spit the pacifiers out and do not like the taste. Medications and gripe water solutions are a hit and miss and often babies with reflux spit it out. As such, there is a need for a solution that soothes a baby for a longer term and provides relief to their stomach problems.

Remote monitoring of an individual, such as a patient, is not new. U.S. Pat. No. 10,136,815 B2 (“815 patent”) is one such example of a patient monitoring system. In the '815 patent, the remote patient monitoring system is used in a hospital environment to monitor a patient in bed in a with more or less stationary devices and monitors, such as medical probes and drug delivery systems, are attached to the patient. The '815 patent also implies monitoring patients in transit, such as then in an ambulance. The attached devices report patient vitals, e.g., heart rate, blood pressure, oxygen saturation, end-tidal CO2, and respiration rate level. The remote monitoring systems transmits the collected patient vitals using to another monitor using wireless technologies, such as Bluetooth or RTM. There are other variations of the remote patient monitoring devices as well that use Wifi and other Bluetooth transmission mechanisms.

One problem with such patient monitoring systems is that they are bulky, require heavier attachments to collect the patient vitals or are used in conjunction with larger stationary devices and stands, such as drug delivery systems and are suitable for adults or individuals in a hospital bed setting. Another problem with the patient monitoring systems is that they use Wifi or Bluetooth to transmit data, which is harmful to the patient. Yet another problem with such patient monitoring systems is that the alarms or alerts are loud (or disturbing). The loud sound used is meant to catch the attention of a caregiver but ends up disturbing the patient or waking the patient from their rest/sleep.

As such, along with soothing the baby and providing relief to their stomach problems, there is also a need to remotely monitor the baby with mechanisms that do not disturb or harm the baby and provide the baby the movement flexibility in their natural setting.

### SUMMARY

A self-heating belly band for wrapping around an infant is provided. The belly band includes a multi-layer fabric construction that captures the infant's body heat and then reuses it to warm the infant's stomach and internal organs thereby providing the infant relief. The belly band also provides pressure on the infant's waist to provide the infant a cuddling, soothing, and comfort feel. A hook and loop fastener strip on top and bottom layers of the belly band allow wrapping and tightness adjustment around the infant's stomach.

The belly band also includes a monitoring system. In one embodiment, the monitoring system includes sensors, a display, and connection to an electronic device, such as a medical monitoring device, or a mobile phone, though a mobile application. The sensors are placed inside the inner layer of the belly band and when they come in contact with the infant, they can measure various vital signs, such as blood pressure, heartbeat, temperature, oxygen level, as well as movement. Connected to a mobile phone through an API, the caregiver is alerted as soon as an emergency occurs or when any of the preselected parameters are exceeded. The API allows the caregiver to set controls and values or select a physician recommended setting. Using the API's user interface, the user can set a threshold for all the vital signs. For example, a temperature threshold for 99 Degrees Fahrenheit can be selected. If the threshold is exceeded, the monitoring system generates an alarm to the authorized user's electronic device.

In another embodiment, the monitoring system includes a belly band monitor and a hub. The belly band monitor includes a circuit board, a plurality of electrical components, including a processor couple to the circuit board, and a transmitter that emits a sound in the ultrasonic range, similar to a Gaston whistle. The hub is located within 150 feet of the belly band and is capable of receiving the transmitted ultrasonic sound.

In yet another embodiment, the monitoring system includes a belly band monitor and a hub. The belly band monitor includes a circuit board, a plurality of electrical components, including a processor couple to the circuit board, and an infrared light transmitter that generates an infrared light. The infrared light generated may be between the range from 400 nm to 1 million nm (or 1 mm). The hub is located within 150 feet of the belly band and is capable of receiving the infrared light transmitted from the belly band. The hub may also be electronically or wirelessly connected to one or more infrared cameras that are capable to detecting infrared light and transmitting the infrared light information to the hub.

The belly band also includes an alarm and a speaker. The alarm provides audible alerts when a vital sign measurement exceeds a normal or preselected criterion, such as a high temperature or heartbeat. The speaker allows the caregiver to play music or simply talk to the baby remotely through their mobile Application. The belly band may also be communicatively connected with the primary care physician to allow immediate response to an emergency event. The alarm can also be through an ultrasonic sound or an infrared light transmitted using its infrared light transmitter or transmitter that transmits ultrasonic sound.

The belly band monitor may be stitched into the belly band such that electrodes that measure the vitals of the baby can touch the baby's skin or the baby's clothing. Alternatively, the belly band monitor may include pogo pins that have a point of contact with the baby to measure the vitals.

### BRIEF DESCRIPTION OF DRAWINGS

The foregoing summary, as well as the following detailed description, will be better understood when read in conjunction with the appended drawings. For the purpose of illustrating the invention, there are shown in the drawings examples that are presently preferred. It should be understood, however, that the invention is not limited to the precise arrangements and instrumentalities shown. In the drawings:

FIG. 1 illustrates a belly band placed underneath an infant that is aligned with the waist and stomach section of an infant according to an embodiment of the disclosed subject matter;

FIG. 2 is cross section of the belly band depicting a multi-layer construction according to an embodiment of the disclosed subject matter;

FIG. 3 depicts the three layers that comprise the belly band according to an embodiment of the disclosed subject matter;

FIG. 4 is a block diagram illustrating a method for positioning and wrapping the belly band across an infant's waist according to an embodiment of the disclosed subject matter;

FIG. 5 depicts a top view and a cross-sectional view of a belly band having sensors and a digital temperature display according to an embodiment of the disclosed subject matter;

FIG. 6 depicts a system that is used to monitor the infant and receive alerts according to an embodiment of the disclosed subject matter;

FIG. 7 depicts another cross-sectional view of a belly band having a monitor according to an embodiment of the disclosed subject matter;

FIG. 8 depicts an assembly view of the monitor used in the belly band according to an embodiment of the disclosed subject matter;

FIG. 9A depicts an isometric front view of the monitor used in the belly band according to an embodiment of the disclosed subject matter;

FIG. 9B depicts a top view of the monitor used in the belly band according to an embodiment of the disclosed subject matter;

FIG. 10A depicts an isometric back view of the monitor used in the belly band according to an embodiment of the disclosed subject matter;

FIG. 10B depicts a bottom view of the monitor used in the belly band according to an embodiment of the disclosed subject matter;

FIG. 11 depicts a cross-sectional view of a belly band having electrodes stitched into the fabric according to an embodiment of the disclosed subject matter; and

FIG. 12 depicts a system that is used to monitor the infant and transmit and receive either audible or infrared signals from the belly band to a hub according to an embodiment of the disclosed subject matter; and

FIG. 13 is a block diagram illustrating a flow chart for monitoring the baby's vital signs and setting an alarm using a mobile electronic device according to an embodiment of the disclosed subject matter.

### DETAILED DESCRIPTION

Reference will now be made in detail to the present examples of the invention illustrated in the accompanying drawings. Wherever possible, the same reference numbers will be used throughout the drawings to refer to the same or like elements.

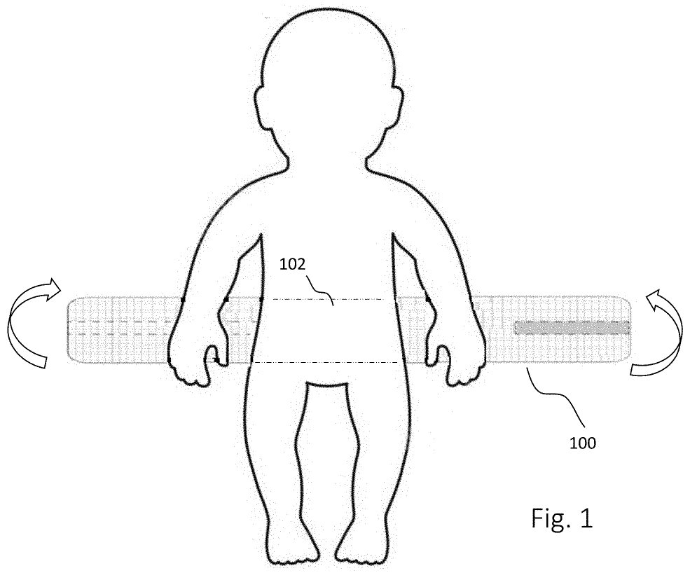

FIG. 1 illustrates a belly band placed underneath an infant that is aligned with the waist and stomach section of an infant according to an embodiment of the disclosed subject matter. The belly band 100 is positioned such that it is able to wrap around the infant's waist line 102 in a snug fit that is not too loose or too tight.

Once wrapped, the fabrics and materials used in in the construction of the belly band the self-heat by capturing the body heat generate by the infant. The materials used include organic cotton front and back that are lined with polyester fleece. The belly band results in both applying pressure as well as heat to the infant's stomach and internal organs thereby facilitating relieving of gas, aiding in digestion and ease constipation, relieving tummy aches and colic symptoms while also helping to achieve better sleep. Since infant have less body fat, they lose heat rapidly, and heating the stomach and organs with the belly bad aids both heat retention as well as calms them to provide that feeling of being inside the mother's womb.

The arrows in FIG. 1 depict the direction of the wrap. The hook and loop fasteners, such as Velcro®, on each side of the belly band allows a snug wrap that can be adjusted by lifting and aligning the hook and loop fastener with the receiving end. Caregivers and parents can adjust the pressure until a comfortable amount of pressure is applied.

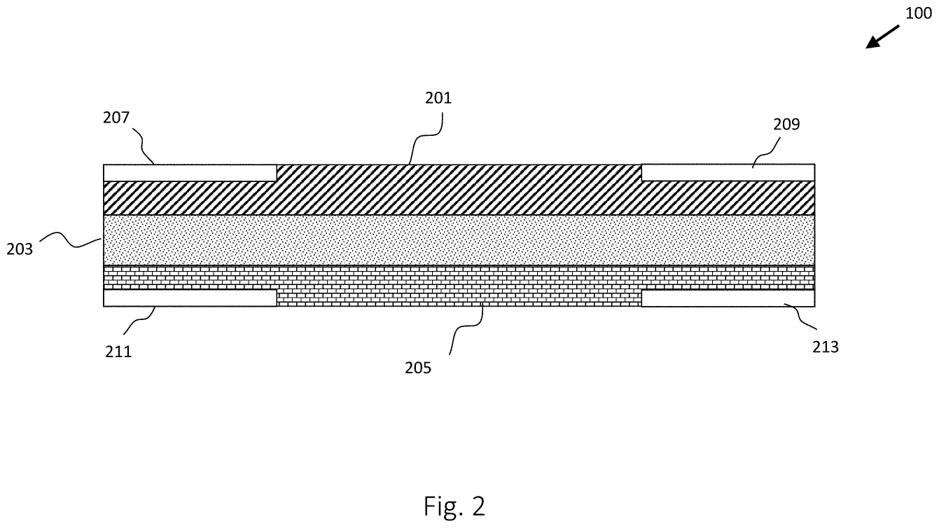

FIG. 2 is cross section of the belly band depicting is multi-layer construction according to an embodiment of the disclosed subject matter. In one embodiment, the belly band is made of three layers. This construction allows heat from the infant's body to be trapped within the fleece fabric and provide extra warmth for the infant.

In one embodiment, the top layer 201 is made from cotton. Other fabrics and textures are also contemplated. The middle or inner layer 203 is made of polyester fleece. This material allows the heat to be trapped and not escape easily. Other heat trapping or insulative materials and fabrics are also contemplated. The bottom layer 205 is also made of cotton.

The top layer 201 includes hook and loop fastener strips 207 and 209. These strips, the attachment means, may be of other hook and loop fastener type or other material that allows attachments by coming into contact with another receiving material and not disengaging without an adequate amount of pressure.

The bottom layer 205 includes receiving strips 211 and 213. In this embodiment, two hook and loop fastener strips and two receiving end strips are shown, however, it is not necessary to have both strips 207 and 209, just one strip is enough. Likewise, it is not necessary to have both receiving end strips 211 and 213, just one receiving end strip is enough. Having two of each facilitates the attachments allowing the caregiver or parent to wrap the belly band from any side they choose.

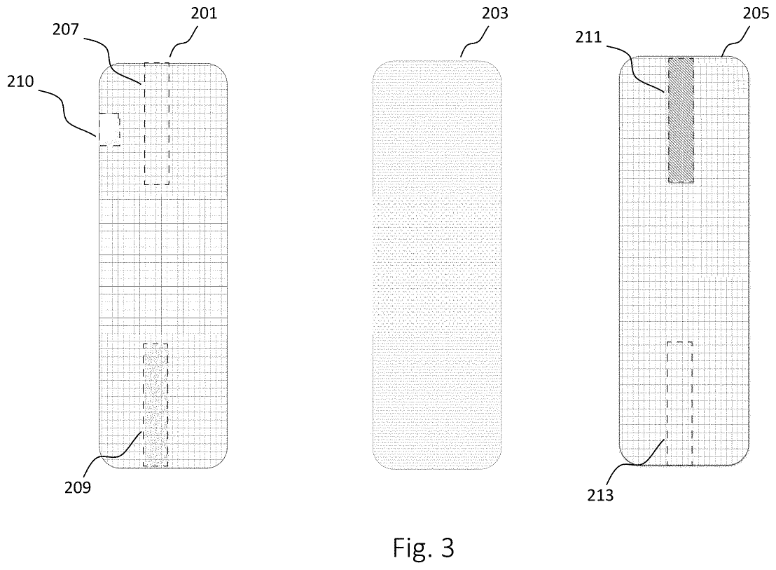

FIG. 3 depicts the three layers that comprise the belly band according to an embodiment of the disclosed subject matter.

The top layer 201 includes hook and loop fastener Strips 207 and 209 that are stitched into the fabric. The top layer also include logo or wash instruction section 210. The middle layer 203 is sandwiched as well as hemmed between the top 201 and the bottom layers 205. The bottom layer 205 has a soft touch fastener 211 and 213. These can be hook and loop fastener receiving strips or alternative soft touch stackable strips that allow in securing the belly band when its wrapped around the infant's waist. The amount of force to open the belly-wrap is minimal for an adult but harder for an infant to undo it him/herself.

The middle layer can be made from a material that traps heat, such as wool, nylon, or a thermally insulated fabric that retains heat. As mentioned earlier, fleece like material is preferred but similar alternatives are contemplated.

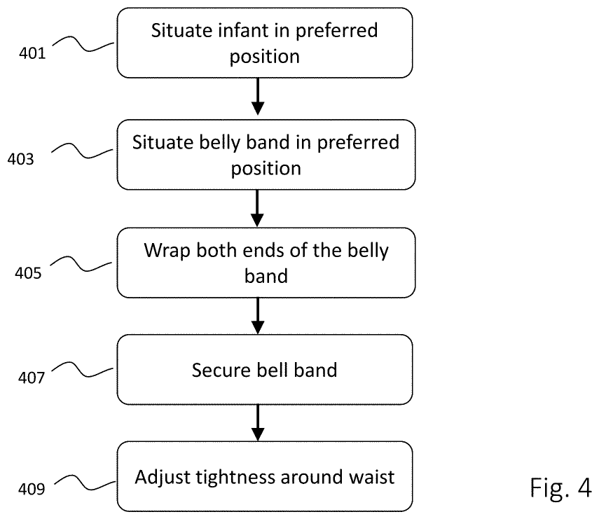

FIG. 4 is a block diagram illustrating a method for positioning and wrapping the belly band across an infant's waist according to an embodiment of the disclosed subject matter.

At Step 401, the infant is positioned such that a belly band can be applied to its waist line. For example, the infant can be sitting up or lying down flat on his/her back.

At Step 403, the belly band is placed in a preferred position. The belly band can be played flat on a bed, crib, or a flat surface or it can be held in hand to apply around a sitting infant's waist. Steps 401 and 403 are interchangeable as long as the infant and the belly band are aligned such that the belly band can be applied around the infant's waist.

At Step 405, the belly band is wrapped around the waist line of the infant such that hook and loop fastener strip 207 or 209 from the top layer 201 comes into contact with the hook and loop fastener receiving end 211 or 213 of the bottom layer 205.

At Step 407, the hook and loop fastener strip from the top layer is pressed upon the hook and loop fastener receiving strip on the bottom layer thereby securing the belly band and preventing it from loosening or coming out without application of upward pressure.

At Step 409, the caregiver may adjust the belly band by applying adequate pressure such that not too much or too little pressure is applied to the infant's waist.

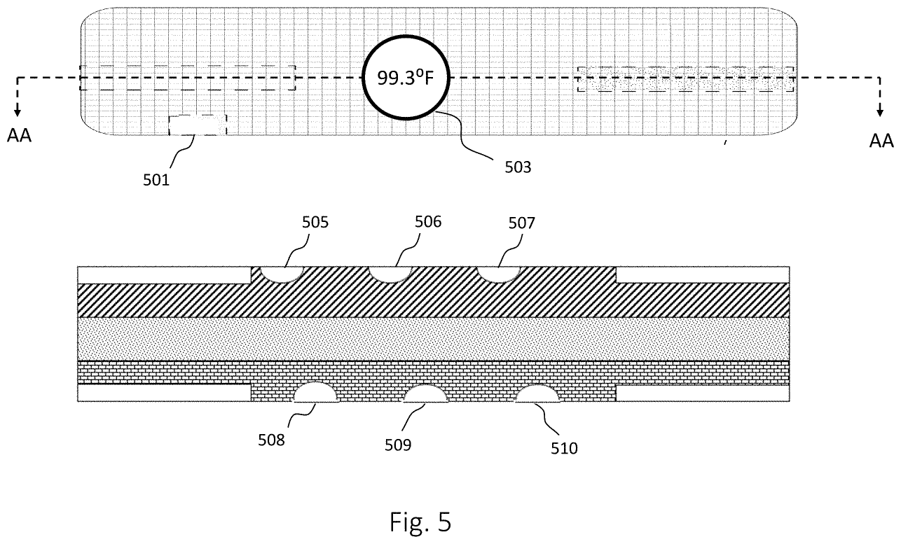

FIG. 5 depicts a top view and a cross-sectional view of a belly band having temperature sensors and a digital temperature display according to an embodiment of the disclosed subject matter.

In one embodiment, the belly band includes a plurality of sensors 505-510. The sensors may be temperature sensors or sensors that monitor the infant's heartbeat, blood pressure, movement, sleep and sleep patterns, humidity, and other vital signs. The sensors 608-610 can be placed in inner layer of the belly band that comes in contact with the infant's body. Other alternative locations for sensor placement 505-507 are also contemplated.

In one embodiment, the sensor is a temperature sensor that measure the body temperature of the infant. The measurements can be taken constantly or at periodic intervals. The sensors detect a fever as well as other temperature conditions such as if the baby is too hot or too cold due to its surroundings, e.g. sometimes due to a thick blanket, or if the baby breaks into a sweat or has the chills.

In another embodiment, the belly band includes a temperature display. The display simply depicts the body temperature of the infant as measured by the sensors. The display can be a digital display of various shapes and sizes. A small battery may be used to operate the display.

In yet another embodiment, a battery-operated alarm may be included in the belly band. The alarm may be set to ring if the temperature measured by the temperature sensor exceeds the allowable range of temperature that is safe for the infant. In another embodiment, the alarm may be programmed with specific desired setting to go off when the temperature reaches a certain level. As will be explained later in detail, the temperature sensor is mounted to a printed circuit board that passes through the bottom cap of a monitor. It makes direct contact with baby's skin.

In another embodiment, the sensor measures the infant's vital signs. These can include heartrate, blood flow, and/or oxygen levels etc. It could also include the baby's breathing rate which can be measured with an accelerometer that is part of the baby monitoring system and connected with the processor as will be explained further. The sensors can also monitor the baby's motion and detect the amount of sleep the baby gets in a certain period of time.

The display may include setting to display additional vital signs measured by the sensor. This alleviates the need to wake up a sleeping infant when it has a cold or fever to measure its vital signs and the caregiver can simply view the attached display and review the displayed data. Likewise, an alarm can be set to sound if any of the vital signs are outside a safe zone or outside a previously determined window.

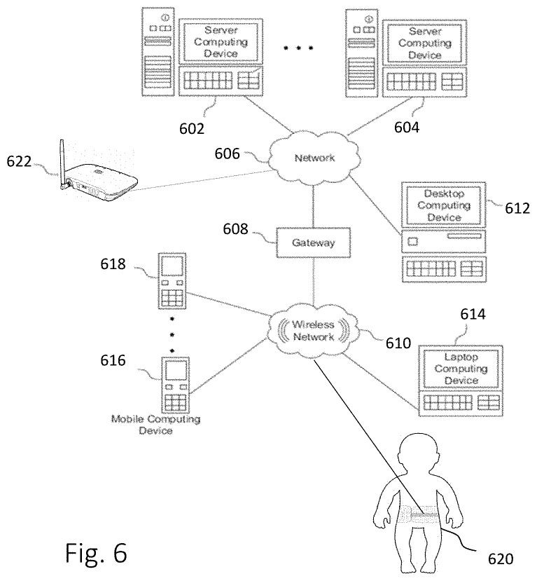

FIG. 6 depicts one embodiment of a system that is used in conjunction with the belly band according to the disclosed embodiments. System 600 is used in connection with the belly band and includes one or more networks, such as a local area networks (LAN) and a wide area network (WAN). The system 600 also includes wireless network 610 that allows devices in the network to wirelessly communicate with each other after pairing and/or other authorization between the devices.

Gateway 608 is configured to connect remote or different types of networks together, as well as client computing devices 612-618 and server computing devices 602-604.

Client computing devices 612-618 may include any device capable of receiving and sending data over a network, such as wireless network 610. Devices 612-618 may include portable devices such as cellular telephones, smart phones, radio frequency-enabled devices, personal digital assistants, handheld computers, tablets, laptop computers, medical monitoring devices, wearable computers and the like. Device 620 is a belly band. Devices 612-618 also may include any computing device that connects to a network using a wired communications medium such as personal computers, multiprocessor systems, microprocessor-based or programmable consumer electronics, network personal computers and the like.

Client computing devices 612-618 also may be web-enabled client devices that include a browser application configured to receive and to send web pages, web-based messages and the like. The browser application may be configured to receive and display graphic, text, multimedia, or the like, employing virtually any web-based language, including a wireless application protocol messages (WAP), or the like.

Client computing devices 612-618 also may include at least one other client application that is configured to receive content from another computing device, including, without limit, server computing devices 602-604. The client application may include a capability to provide and receive textual content, multimedia information, or the like. The client application may further provide information that identifies itself, including a type, capability, name, or the like. In one embodiment, client devices 612-618 may uniquely identify themselves through any of a variety of mechanisms, including a phone number, mobile identification number (MIN), an electronic serial number (ESN), mobile device identifier, network address, such as IP (Internet Protocol) address, media access control (MAC) layer identifier, or other identifier. The identifier may be provided in a message, or the like, sent to another computing device.

Client computing devices 612-618 may also be configured to communicate a message, such as through email, short message service (SMS), multimedia message service (MMS), instant messaging (IM), which may include both video and audio messages to another computing device. Belly band 620 may also receive messages through the network or wireless network from an authorized user.

Network 606 is configured to couple one or more servers depicted in FIG. 1 as server computing devices 602-604 and their respective components with other computing devices, such as client device 612, and through wireless network 610 to client devices 614-618. Network 606 is enabled to employ any form of computer readable media for communicating information from one electronic device to another. Network 606 also may include the Internet in addition to local area networks (LANs), wide area networks (WANs), direct connections, such as through a universal serial bus (USB) port, other forms of computer-readable media, or any combination thereof. On an interconnected set of LANs, including those based on differing architectures and protocols, a router acts as a link between LANs, enabling messages to be sent from one to another. Network 606 may include any communication method by which information may travel between computing devices. Additionally, communication media typically may enable transmission of computer-readable instructions, data structures, program modules, or other types of content, virtually without limit. The above-mentioned system may allow communications between the belly band and pharmacy, caregiver, and user's electronic devices, such as a mobile phone, tablet, medical monitoring device etc. to send alerts.

A local hub 622 is also connected to computing devices 602-604 as well as client device 612, client devices 614-618, and Belly band 620, through wireless network 610 or Network 606. The local hub 622 accesses the data from the Belly band 620 and transmits the data to client devices or other remote devices. For example, the hub 622 can obtain an infant's temperature that was read through the Belly band 620 and then transmit it to an authorized mobile device or computer.

In yet another configuration, not shown, cloud computing may be used. In such configuration, a cloud computing environment may provide computing devices 602-604 and client devices 614-618 with one or more resources provided by the network environment. The clients and devices may be in communication with the cloud over a network. Clients may include, e.g., thick clients, thin clients, and zero clients. The cloud may also include back end platforms such as servers or data centers. The cloud may be public, private, or hybrid.

In another embodiment, an authorized member of the system, such as a caregiver or parent, can download an Application on their mobile phone and connect to the system through the API thereby allowing access to the infant's vital signs and other measurements made by the sensors. Further, using the Application, the Parent or Caregiver can also push alerts, such as an alarm, musical tone, or just speak to the infant from a remote location.

The networked connectivity as mentioned above allows remote monitoring of the infant as well as receiving alerts in the event of an emergency or in the event any of the sensors obtain a measurement that exceeds a preselected parameter, such as for heartrate, blood pressure, temperature, or other vital signs. The parent or caregiver may also set alerts to their mobile phone when a motion is detected, such as when a sleeping baby finally wakes up. This allows the parent or caregiver do other work while the infant is sleeping and not be tied to a display monitor yet attend to the infant as soon as they wake up.

Also as mentioned above, the bell-band includes a plurality of sensors. The network environment allows the sensors to directly communicate through WIFI or Bluetooth to an electronic device, such as a mobile phone. In one embodiment, the belly band is paired with the electronic device. Standard pairing methods through Bluetooth, WIFI and others are used to pair it with the electronic device. A password and User ID may also be required to complete the pairing of the devices. The method may also involve, connecting to the local WIFI and restarting and rebooting one or both devices.

In yet another embodiment, the belly band may be communicatively connected to the infant's primary care physician. If an emergency occurs, such as sudden rise or drop in temperature, severe vital sign issues, then an emergency alert may be sent to the primary care physician directly along with relevant data, GPS location, and contact information of the caregiver such the emergency is attended to immediately. In many instances the caregivers may not be knowledgeable of an emergency event and the belly band assists in getting the right help in real-time.

The preferable connection between the belly band and another device, such as hub or any other electronic device, is encrypted and also uses a safer mode of communication, such as sound, ultrasonic sound, light, or infrared light instead of Wi-Fi signal. The is specific to the data being sent or received at the belly band location. Other devices that are farther away from the belly band, such as a sound receiver or a camera that connects to the hub can use a variety of communications methods, including Wi-Fi since it is located farther away from the infant. All communications are encrypted and authorized such that only devices and people with authorized access can send or receive data.

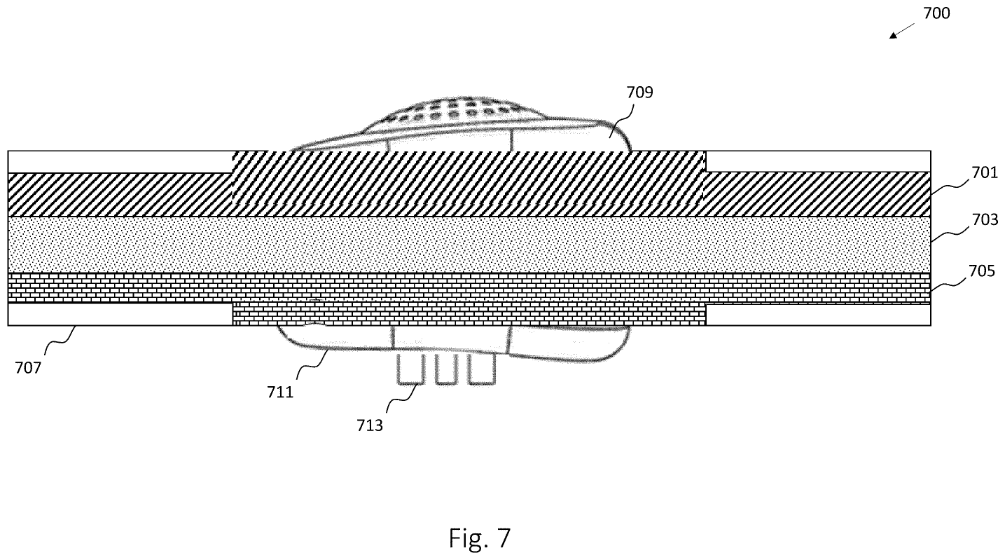

FIG. 7 depicts another cross-sectional view of a belly band having a monitor 701 according to an embodiment of the disclosed subject matter. The belly band 700 includes a plurality of fabric layers, e.g., top layer made of cotton 701, middle or inner layer 203 made of polyester fleece, and bottom layer 705 made of cotton. The fabric includes a removable mechanism, such as hook and loop fastener or touch fastener 707 on either end.

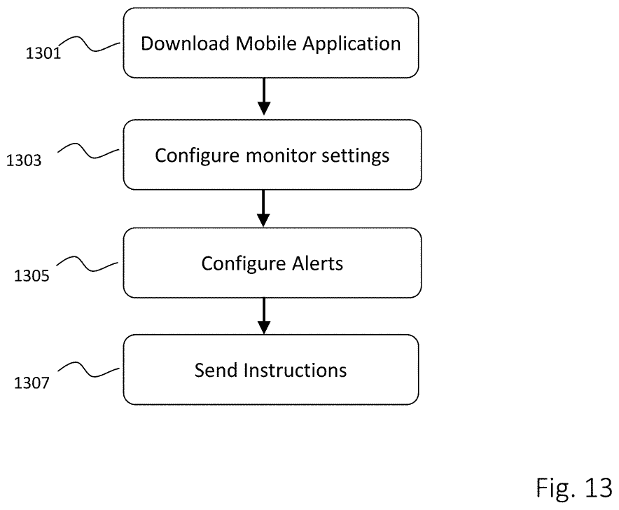

The belly band also includes a monitor that has a top housing 709 and a bottom housing 713. The top housing includes a speaker and the bottom housing includes a plurality of pogo pins 713. The speaker is used to sound an audible alarm or alert when needed. The setting of the audible alert is discussed further in the FIG. 13 below.

In one embodiment, the monitor is fastened and stitched within the three layers 701-703 of the belly band fabric. The stitched in monitor is fastened such that it cannot be easily removed from the belly band.

In another embodiment, the inner layer 705 of the belly band a pouch-like opening. The monitor can be removably placed inside the pouch-like opening and fastened with hook and loop fastener, touch fastener, a button, or a press fastener. In this position, the pogo pins are inward thereby touch the baby's clothing or skin when the belly band is fastened. The pogo pins are metallic electrodes that are capable of reading the baby's vitals by coming in contact with either the baby's skin or coming in contact with the layer of clothing worn by the baby.

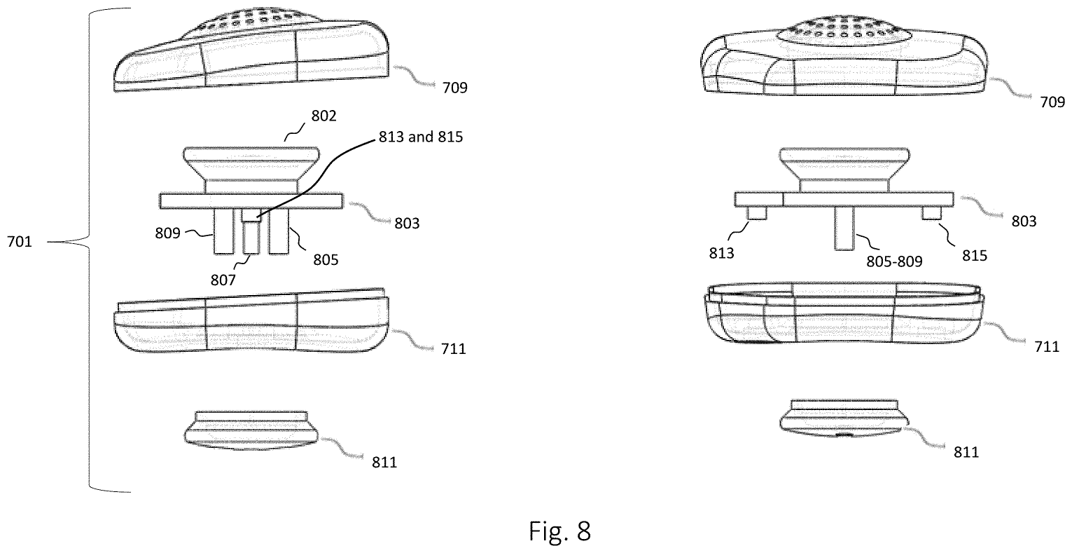

FIG. 8 depicts an assembly view of the monitor used in the belly band according to an embodiment of the disclosed subject matter. FIG. 8 is also a detailed view of the monitor described in FIG. 7. The assembly view depicts the top housing. In some embodiments, the top housing includes a speaker cover. In other embodiments, the monitor uses other methods of sounding an alarm or alerting a caregiver, such as ultrasonic sound or an infrared light. As such the covers vary depending on the mode of transmission.

The middle section of the monitor includes a speaker 802, printed circuit board 803, electro cardiogram or ECG leads or electrodes 805 and 809, and thermal couple 807 for reading temperature. The middle section of the monitor also includes power pins 813 and 815.

The bottom section 711 include a bottom cap 811. The bottom cap 811 has an opening that allows the ECG leads/electrodes 805 and 809, thermal couple 807, and power pins 813 and 815 protrude outside the cap.

The speaker 802 is used by the monitor to create audible sounds. These sounds can be preselected sound or can be chosen by the user. The audible sounds generated can be a regular audible sound or an ultrasonic sound ranging between 20 Hz to 200 Mhz. The user may also download a sound through the mobile application and use it as an alarm or alert sound. The user may also choose different types and decibels of sounds for different indications. For example, the user may select a certain sound to alert them if the temperature of the baby above the threshold and another sound if the heartbeat is above a threshold. The speaker can also be used to play soothing music or white noise to calm the baby or put the baby to sleep.

The printed circuit board (PCB) 803 includes a plurality of electrical components that include a microprocessor and a memory that is connected to the microprocessor. The microprocessor analyses the baby's vitals along with any instructions provided by the user/caregiver or a physician. The microprocessor is communicatively coupled to the ECG leads or electrodes 805 and 809, thermal couple 807, and power pins 813 and 815. The microprocessor also instructs electrical components to perform certain functions to take the baby's vitals. For example, it can periodically instruct the ECG leads or electrodes 805 and 809 to perform an electro cardiogram of the baby. The intervals at which the vitals are taken can be predetermined, or selected by the user or a physician. The user or a physician can also push an instruction at any time that will result in the microprocessor activating the ECG leads or electrodes 805 and 809 to take the ECG.

The thermal couple 807 acts as a thermometer and is used to detect the baby's temperature. Similar to the ECG, the temperature can be taken periodically or as directed by the user.

The power pins 813 and 815 are used for charging the monitor. The monitor includes an internal rechargeable battery that can be recharged through by placing the power pins in contact with a power outlet or the monitor docking device that connects a power outlet with the pins once docked.

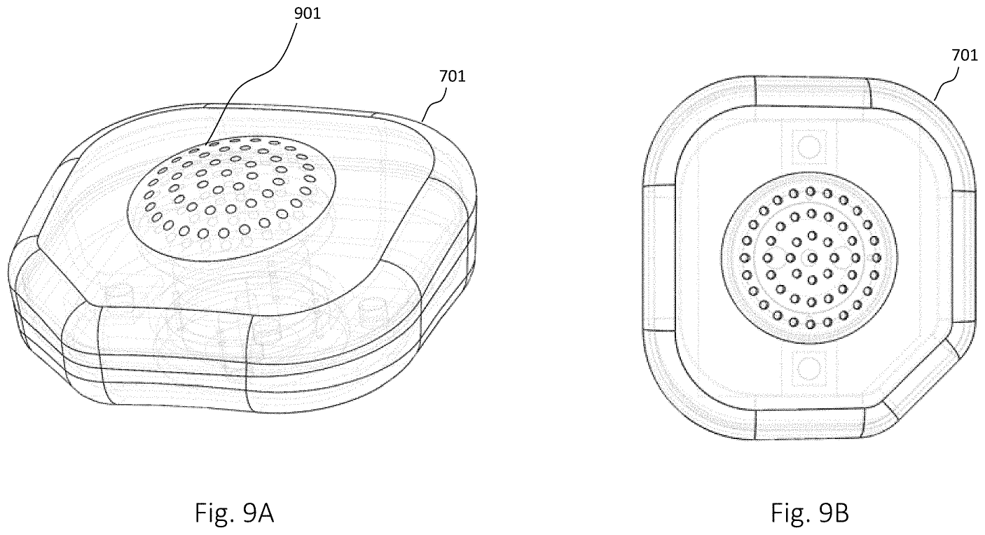

FIG. 9A depicts an isometric front view of the monitor used in the belly band according to an embodiment of the disclosed subject matter. FIG. 9B depicts a top view of the monitor used in the belly band according to an embodiment of the disclosed subject matter. Both figures show the top section of the monitor housing. The speaker cover 901 is located above the speaker that is located underneath the cover and inside the monitor 701.

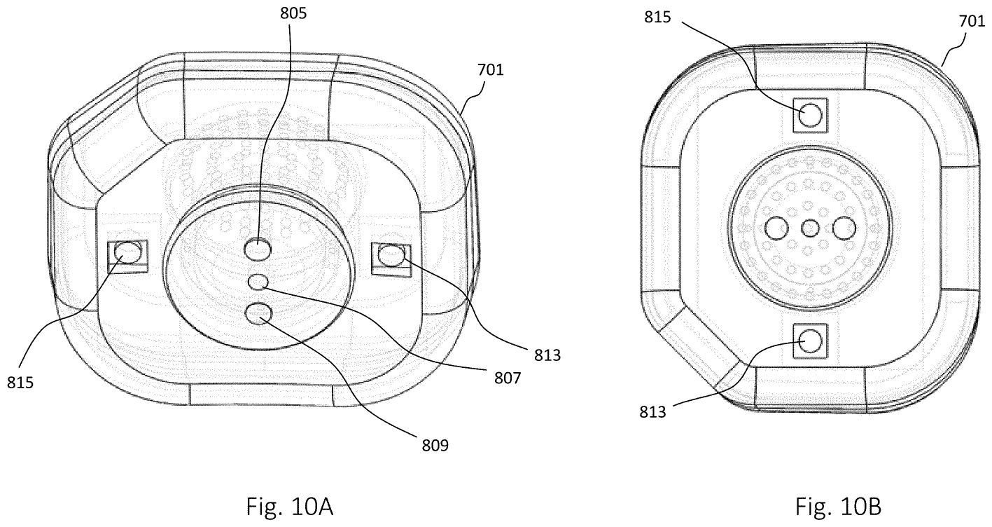

FIG. 10A depicts an isometric back view of the monitor used in the belly band according to an embodiment of the disclosed subject matter. FIG. 10B depicts a bottom view of the monitor used in the belly band according to an embodiment of the disclosed subject matter. The ECG leads or electrodes 805 and 809 and the thermal couple 807 is shown protruding outside the bottom cap of the monitor 701. The power pins 813 and 815 also protrude outside, however, they are located in a recessed section in the bottom side of the monitor. The monitor can be inserted in a charging station where the power pins 813 and 815 come into contact with an electrical charge. The power pins 813 and 815 are connected to the rechargeable battery included inside the monitor and are used to recharge the battery.

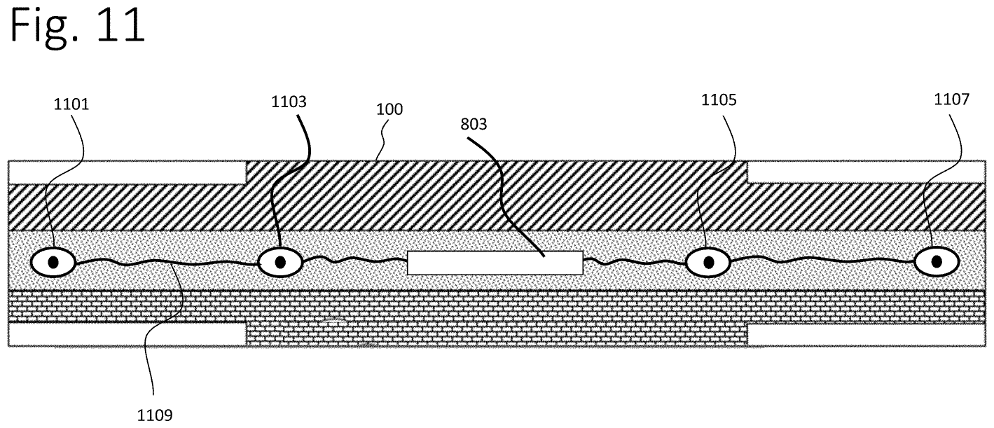

FIG. 11 depicts a cross-sectional view of a belly band having electrodes stitched into the fabric according to an embodiment of the disclosed subject matter. In this embodiment, the belly band 100 includes a plurality of electrodes 1101, 1103, 1105, and 1107 that are stitched in-between the fabric layers 201, 203, and 205 (referring to FIG. 1) such that a portion of the electrodes is visible outside the inner layer 205. The electrodes come into contact with the baby's skin or the baby's clothing and are used for determining the baby's vital signs. Likewise, thermocouples can also be disbursed and stitched within the fabric layers to obtain the baby's temperature. Although four electrodes are shown, the number and placement can vary.

The electrodes 1101, 1103, 1105, and 1107 are connected to each other and to the printed circuit board 803 through wiring 1109. The microprocessor in the printed circuit board 803 obtains the ECG of the baby through these electrodes and processes and analyses the information obtained. The information is then transmitted to the user or caregiver.

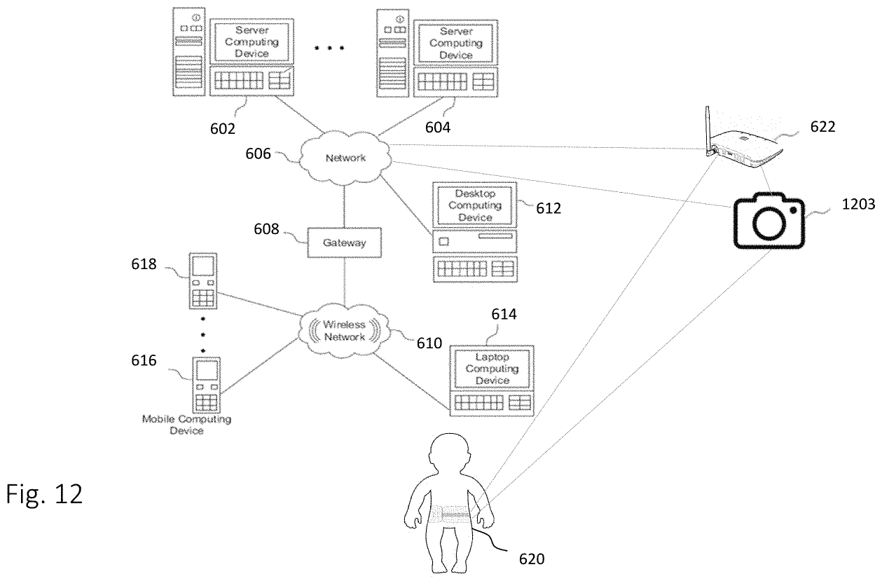

FIG. 12 depicts a system that is used to monitor the infant and transmit and receive either audible or infrared signals from the belly band to a hub according to an embodiment of the disclosed subject matter. The embodiment is similar to FIG. 6 and includes an addition of an Infrared (IR) Camera 1203. When an IR transmission method is used, the monitor includes a transmitter that can transmit IR light. The IR light is read by the IR camera 1203. The IR camera is connected to the hub 622 as well as the network 606. In this embodiment, the belly band or the monitor does not have any Bluetooth or wireless capability since such capability generates radiation at a higher level that is harmful to the baby. The IR light does not harm the baby nor can it be seen by the human eye. The IR light ultrasonic is transmitted from the belly belt in response to a vital sign exceeding a predetermined threshold.

In another embodiment, as mentioned above, the system includes an ultrasonic sound receiver. Alternatively, the ultrasonic sound received can be a part of the hub 622. The ultrasonic sound receiver is connected to the hub and in close vicinity with the belly band, preferably within 150 ft for better reception. The ultrasonic sound received receives the ultrasonic sound transmitted from the belly belt in response to a vital sign exceeding a predetermined threshold. This embodiment also does not include Bluetooth or WIFI capability.

Although sounds and light have been described above for modes of communication from the belly band, the invention is not so limited and other safer modes, such as other non-Wi-Fi modes of communication, from the belly band are also contemplated.

FIG. 13 is a block diagram illustrating a flow chart for monitoring the baby's vital signs and setting an alarm using a mobile electronic device according to an embodiment of the disclosed subject matter. At 1301, the user of the system can download a mobile application on their mobile electronic device. The user can be a parent, caregiver, physician, or an authorized friend, family member or nanny. The mobile application allows connection with the processor of the monitor that is located in the belly band.

At 1303, the user configures the setting using the mobile application. These setting include thresholds for vital signs, intervals to check vital signs, and a list of authorized individuals that can receive an alert.

At 1305, the user can select from a predetermined list of alarm and alerts settings and sounds or configure their own alerts. For example, configuration setting include selecting multiple set of individuals to receive an alert, setting up first and second alerts, selecting alerts sounds and decibel levels, and selecting audible or visual alerts.

At 1307, the user can send instructions to the processor to check a certain vital on demand. The user can change intervals of vital checks or set timers.

It will be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention as defined by the appended claims.

We claim:

1. A belly band for wrapping around an infant child to soothe the child and relieve the child of gas, indigestion, colic symptoms, the belly band comprising:
a top layer having one or more hook and loop fastener strips;
a bottom layer, wherein the top layer and the bottom layer are hemmed together, wherein the bottom layer includes one of more hook and loop fastener receiving strips, wherein when the top layer's hook and loop fastener strip comes in contact with the bottom layer's hook and loop fastener receiving strip, the belly band is secured such that is cannot be opened until a pressure is applied in the upward direction to detach the top layer's hook and loop fastener strip from the bottom layer's hook and loop fastener receiving strip; and
a middle layer sandwiched in-between the top and the bottom layer, wherein the middle layer is made from a fleece like material that can capture the body heat from the infant, store the heat, and then apply the heat back to the infant in the areas in which the belly band comes in contact with the infant.

2. The belly band of claim 1, further comprising a plurality of sensors that are stitched within either the top layer, the bottom layer, or both top and bottom layers, wherein the sensor measures the temperature or a vital sign of the infant.

3. The belly band of claim 2, wherein the sensor detects the temperature of the infant.

4. The belly band of claim 3, further comprising a temperature display, wherein the temperature sensed by the temperature sensors is displayed on the temperature display.

5. The belly band of claim 4, further comprising an alarm, wherein, the alarm is sounded when the temperature exceeds or decreases from an allowable range of temperature, when such increase or decrease is detected by the temperature sensor.

6. The belly band of claim 3, wherein the sensor measures the infant's heartbeat.

7. The belly band of claim 3, wherein the sensor measures the infant's oxygen level.

8. The belly band of claim 3, wherein the sensor measures the infant's blood pressure.

9. The belly band of claim 3, wherein the sensor detects the baby's movement.

10. The belly band of claim 2, further comprising a communications module that provides connectivity to a remote mobile device through a mobile application, wherein a parent or caregiver is able to download the mobile application and receive data measured by the sensors.

11. The belly band of claim 10, wherein the parent or caregiver is alerted on their mobile phone in the event of an emergency or is a measure criterion exceeds an allowable or preprogrammed criterion.

12. An infant monitoring system comprising:
a belly band for wrapping around an infant child
a monitor having a processor and a non-volatile memory;
a hub communicatively connected to the monitor, wherein the hub is configured to receive data transmitted from the monitor and transmit the received data to a connected device; and
a mobile electronic device having a display and a user interface, wherein the mobile electronic device is communicatively connected to the hub, wherein the mobile electronic device displays the data transmitted by the hub on its user interface.

13. The infant monitoring system of claim 12, wherein the belly band comprises
a top layer having one or more hook and loop fastener strips;
a bottom layer, wherein the top layer and the bottom layer are hemmed together, wherein the bottom layer includes one of more hook and loop fastener receiving strips, wherein when the top layer's hook and loop fastener strip comes in contact with the bottom layer's hook and loop fastener receiving strip, the belly band is secured such that is cannot be opened until a pressure is applied in the upward direction to detach the top layer's hook and loop fastener strip from the bottom layer's hook and loop fastener receiving strip; and
a middle layer sandwiched in-between the top and the bottom layer, wherein the middle layer is made from a fleece like material that can capture the body heat from the infant, store the heat, and then apply the heat back to the infant in the areas in which the belly band comes in contact with the infant.

14. The infant monitoring system of claim 12, further comprising
a sound transmitter for transmitting sounds, wherein the sound transmitter is electronically coupled to the monitor's processor; and
a sound receiver connected to the hub, wherein the monitor's processor, upon detecting that an infant's vital sign is above or below a predetermined threshold, instructs the sound transmitter to transmit a sound which is then received by the hub.

15. The infant monitoring system of claim 14, wherein the sound transmitted is an ultrasonic sound.

16. The infant monitoring system of claim 14, wherein the sound transmitted is an audible sound.

17. The infant monitoring system of claim 14, wherein the sound is transmitted when the infant's electrocardiogram is above or below a predetermined threshold.

18. The infant monitoring system of claim 13, wherein the monitor is placed in a pouch stitched into the bottom layer of the belly band such that a plurality of electrodes from the monitor protrude outwards and come into contact with the infant's body when the belly band is wrapped around the infant.

19. The infant monitoring system of claim 12, further comprising a light transmitter for transmitting light, wherein the light transmitter is electronically coupled to the monitor's processor; and
a camera, wherein the camera detects a light transmitted by the light transmitter, wherein the camera is communicatively connected to the hub, wherein the camera analyses the information contained within the light and communicates the information to the hub.

20. The infant monitoring system of claim 19, wherein the light emitted is an infrared light between the range from 400 nm to 1 million nm (or 1 mm).

21. The infant monitoring system of claim 13, further comprising a plurality of electrodes disbursed in the bottom layer of the belly band such that the plurality of electrodes come into contact with the infant's body when the belly band is strapped around, wherein the electrodes are used for performing an electro cardiogram.

22. The infant monitoring system of claim 12, wherein the mobile electronic device is capable of downloading a mobile application that allows communication between the mobile device and the monitor's processor.

23. The infant monitoring system of claim 22, wherein the mobile application has an interface that can be used by a user to configure an alert and configure a vital sign threshold.
